(19) United States
(12) Patent Application Publication
(10) Pub. No.: US 2002/0169364 A1
(43) Pub. Date: Nov. 14, 2002

(54) Brainstem and limbic disorder (BALD)

(76) Inventors: William E. Baumzweiger, Studio City, CA (US); Kathleen L. Hannan, Orlando, FL (US)

Correspondence Address: WILLIAM E. BAUMZWEIGER, MD, 18399 VENTURA BLVD., SUIE 245, TARZANA, CA, 91356, US

(21) Appl. No.: 09/503,656

(22) Filed: Feb. 14, 2000

(51) Int. Cl. A61B 5/00

(52) U.S. Cl. 600/300; 128/898

(57) ABSTRACT

A newly discovered diagnostic protocol that localizes a disorder of the human body to the brainstem and also possibly the limbic system of the human body with a clinically proven protocol for treatment of the disorder. Treatment for dysfunction of other parts of the body associated with the brainstem disorder is outlined. This diagnostic protocol and treatment protocol has been designated with the acronym BALD. This discovery covers neurological disorders caused by toxic substances, by infection, and by a combination of toxic substance and infection causative factors. The diagnostic protocol outlines a step by step procedure for determination of the BALD syndrome and the treatment protocol outlines a step by step procedure for the physician to treat the syndrome as well as for clinically successful treatments of other parts of the body associated with the BALD syndrome.

### FIELD OF THE INVENTION

This invention relates to neuro-toxicity and disorders of the central nervous system

Specifically disorders of the brain stem and limbic system of the brain are localized in diagnosis then a specific treatment protocol is outlined.

The acronym BALD is used to refer to the brainstem and limbic system disorder in this specification.

### BACKGROUND

The central nervous system is defined as the brain and spinal cord. Associated with the brain are twelve Cranial Nerves which enervate specific areas of the body. These cranial nerves are associated with the brain stem. The brain stem is the area of the brain where the spinal cord connects with the brain itself. The limbic system of the brain refers to a ring of structures that form a border around the brainstem and corpus callosum of the brain.

There are a number of recognized disorders of the central nervous system. These disorders include seizure disorders such as epilepsy, vascular disorders producing headache, and degenerative disorders. CNS degenerative disorders include inherited or degenerative diseases which includes Amyotrophic Lateral Sclerosis (ALS) and nutritional degenerative disease such as vitamin deficiencies and alcoholic abuse. Extrapyramidal syndromes are disorders which arise from lesions principally in the basal ganglia. Parkinsonism is another such degenerative disease which, like extrapyramidal syndrome, involves movement disorders. There are also autoimmune disorders such as Myasthenia Gravis which affect the neuromuscular junction, and other immune disorders which affect the Myelin insulation of the nerves, or their ability to produce critical brain chemicals.

It is well known that the brain is vulnerable to injuries. It is less well known that the deep structures of the brain are more vulnerable to traction than crush or torsional injuries. Further, the Nervous System is vulnerable to more than just trauma and acute infection. The brain demonstrates vulnerability to changes in levels of glucose, ammonia and other simple molecules. It demonstrates exquisite responsitivity to deprivation of Oxygen and glucose. The brain reacts dramatically to solvents used in mechanical repair areas, to carbon monoxide fumes such as from propane combustion and to toxins such as phenol and toluene. In addition, it demonstrates immune reactivity as in the disease cerebral lupus, to chronic ischemic vascular disease as in as in diabetes, to autoimmune illness as in certain types of cancers, and to oxidative stress from liver failure and concomitant excess of ammonia.

The result in the brain of these combinations of causative conditions, with a final single or ensemble of sub clinical insults is, both central and peripheral neuropathy as well as eventual muscle weakness and limb numbness and pain. Eventually the individual develops a set of nervous manifestations characterized by headaches, fatigue, sleep difficulties, heart rate instability, and gastrointestinal disturbance.

The victim of the BALD syndrome described herein develops symptoms other than at the brain and immune system, such as rashes from immune problems and infections, chronic multiple fungal infections, pulmonary, lung and peripheral vascular disease, as well as premature aging on a cellular level. There can be increasing autonomic instability, with body temperature instability, abnormalities of blood flow to organs and skin, and abnormal salivation and sweating. The diagnostic and treatment protocols will examine all these symptoms.

All types of diseases, including the neurological diseases are more common in the urban environment, where there is concomitant exposure to industrial and environmental toxic, and polluting substances. People are more frequently developing extreme vulnerability to everyday fumes and substances that many other people tolerate. This is being named Multiple Chemical Sensitivity. There appears to be an increased risk for chronic diseases, including Diabetes, Asthma, and also the Neurological/Neurotoxic diseases. It is known that among the black population, babies who were conceived and had embryonic fetal life in urban America are at more than twice the risk to be stillborn. This is of source neurological death in utero. It is clearly related to the multiple sources for neurotoxicity in our urban environment.

A core concept of this invention is that there can be chronic inflammation in neuronal tissue, and loss of adequate Oxidation/energy from oxidative metabolism in critical neural control centers in the brain. Further, this invention seeks to demonstrate that BALD arises from inflammatory immune abnormalities, degradation of connecting axons in the brain, chronic infection especially by fungi and viruses with release of sequestered neuro toxins. This leads eventually to the development of autoantibodies, and chronic electrical instability in neural circuits. Prebirth children would be of course at greatest risk for this type of pathological process. Crib death has just been shown to be the result of fungi acting on the fire retardant in the crib's bedding, causing the production of three types of poison gas. This causes asphyxia in small babies, whose nervous systems are not mature enough to fight off the toxins which are in the environment.

In the brain, because of its complexity the neurological and neuroimmune mechanisms, a comprehensive yet focused approach to the core disease mechanisms is necessary. Further the potential for a very insidious onset of signs and symptoms due to the slow accumulation of damage makes sophisticated testing and pathological analysis essential.

However, as highlighted by this invention some of the basic pathological mechanisms are now understood, and the treatment approach follows naturally from the pathology. One central mechanism is the condition wherein Oxygen does not sufficiently diffuse from the arterial blood to neurons which need Oxygen to transform Carbon Monoxide which is a neuro transmitter into more harmless Carbon Dioxide.

Another of the most central of these mechanisms is called excitotoxicity, which is due to breakdown of control over flow of Calcium ions into neurons which leads to neuronal death. This tendency toward excitotoxicity makes the brain and spinal cord vulnerable to process such as reactivation of intra neuronal neurotropic viruses which leads to intracellular damage through mechanisms which are at present unspecified, but much inflammatory damage in the neuron is mediated by Calcium. It is clear that the excessive influx of Calcium into the neuron up regulates a host of secondary messengers that create a number of inflammatory problems. (Baumzweiger, 1998)

It was first noticed that Calcium had a special role in the transmission of information in 1977. (Baumzweiger, 1999) This was first noticed in ALS, where antibodies against L-Calcium channels were found. (Baumzweiger, 1999) This process has been called excitotoxicity. However, there is a second set of problems that arises with this type of damage called oxidative stress, from a combination of diminished availability of Oxygen, reduced ability to use Oxygen to make critical energy storage and biochemical molecules that repair tissue such as methionine which participate in defending healthy tissue. “Free Radicals”, which are abnormally electrically charged molecules are one of the results. The liver usually shows the stress from dealing with these processes.

This type of metabolic and neuroimmune stress causes a number of problems to the central nervous system, the liver, kidneys, and to the immune system itself. For the nervous system oxidative stress appears to cause damage to the acetylcholine receptors, especially the nicotinic receptors associated with motivation, concentration and mental control. (Baumzweiger National Academy of Science Presentation to the institute of Medicine, Oct. 16, 1999)

The combination of these two problems of toxicity and infectious disease, which play off against each other makes many processes in the nervous system able to reactivate sequestered live virus, fungus and bacteria. When these organisms are captured and sequestered by healthy brain cells, they are generally stopped from reproducing and re-infecting neighboring cells. When they break out, which occurs in multiple sclerosis, there is reactivation of these microbes with inflammatory tissue destruction.

Through advanced Lymphocyte testing, viral antibody testing, fungal antibody testing, venous partial pressure of oxygen tests, and testing for autoimmunity, there appears to be a definable condition where the nervous and immune systems meet in the brain stem and limbic system of the brain (BALD). Further there is a tendency towards BALD neurodysimmunity which is a unique mixture of neural dysfunction, immune suppression and multi system autoimmunity which is not seen in any other disease entity.

### SUMMARY OF THE INVENTION

There has been a long felt need for diagnosis and treatment of the above described problems of neuro toxicity. Because the problems involved in localizing pathology to separate areas of the central nervous system the signs and symptoms of the present invention are subtle and often have been dismissed or overlooked by the physician. Thus, one principal object of the present invention is to delineate the clinical signs and symptoms and laboratory findings resulting from priortrauma or toxic exposure then worsened by specific toxic substances, trauma, or infectious as well as autoimmune disease. Understanding this two pronged pathogenesis will result in changes in medical practice, with a focus on the pathologies due to toxic substances in the modern environment.

It is little recognized that the nervous system can and does react with repeated combinations of toxic exposure and infectuous disease, with the onset of tissue damage, even when the individual insults are sub threshold. A protocol for the diagnosis and treatment of the resulting clinical syndrome with reduction of damage to specific areas of the deep brain systems is desirable. With this invention, it will be possible to assess and treat the damage and the vulnerability to further damage from such combinations, as well as their complications such as reactivation of neurotropic virus, and subsequent excitoxic damage. (Baumzweiger, 1999) A similar tendency toward complexity can be seen in the newly elucidated genetic etiology of schizophrenia and the participation Chlamadia appears to play in heart disease, and which Helicobacter plays in the patho-genesis of peptic ulcers.

An extreme case of central nervous system dysfunction caused by toxic substance would be victims of so called nerve gas used in actual wartime environments. It is a principal object of the present invention to localize the central nervous system disorder that is caused by such toxic substances and to describe a treatment protocol for the disorder.

Up until the advent of the present invention, it has not been possible to clinically localize BALD central nervous system disorders to where they originate, that is to specific areas of the brain stem, the basal ganglia connected to the brainstem, the thalamus which coordinates the rest of the brain by referencing these deep structures, and the axonal transmission pathways to the rest of the brain from the brainstem, basal ganglia, and limbic system. Localization of CNS disorders to one or more of these structures is another principal object of the present invention.

Many disorders related to the brainstem and limbic system have been mis-diagnosed in the past. A few, such as Parkinsons Disease and Acute Brainstem Encephalitis are well known, but the brainstem and limbic system area has not leant itself to classic techniques of neurological localization. It has had less attention than it deserves. Chronic damage to and inflammation of these areas of the CNS are still not accepted by many physicians, although they are known and accepted by specialists in this area. Further, damage to the brainstem and limbic system is hard to discern, and a very careful, systematic, anatomically based and highly technical approach is needed. It is the principal objective of the present invention to correct this situation not only with a diagnostic protocol but with a treatment protocol as well.

The primary purpose of the present invention is to localize and discern the underlying mechanisms which are driving the chronic signs and symptoms, and then treat the resulting disorders. As with all illnesses, eliciting the relevant elements of medical history is essential. As with all illnesses, a history of infections which cause neurological symptoms, a history of nervous system trauma, a history of exposure to environmental, industrial, or wartime toxic or poisonous substances is also essential. This invention involves a diagnostic protocol involving the patient history then a clinical examination to localize the area of nerve tissue damage to the brain stem and limbic systems.

Corroborative laboratory tests and other tests can be run as well.

The damage which charged particles can inflict on tissues is typified by the Calcium ion entering neurons and other cells. Calcium ion is the most important signaling ion in the nervous system. With an excess of Calcium in the neurons too little learning occurs or proper response to the environment does not take place, and the subject becomes somnolent. It is one object of this invention to provide a treatment protocol for such a condition which is aimed at reducing Calcium entry into the neuron, decreasing the excess excitability in neural circuits, reducing the activity of intracellular virus and fungus infections, as well as mdication to deal with intencurrent inflammatory infections such as Mycoplasma Fermentas Incognitas, Herpesvirus 2 and 6, Cytomegalovirus, and fungal infections.

The present method of diagnosis and treatment of the BALD syndrome is aimed at reduction of brainstem and limbic system dysfunction in two critical ways. First it would reduce the damage from Calcium ion in the nervous system no matter what the cause. Secondly it would aim to repair the damage caused by prior excitotoxic damage to neurons, free radical damage to proteins, as well as inflammatory and infectious damage to critical bodily tissues.

### DETAILED DESCRIPTION OF THE INVENTION

This invention consists of 1) a diagnosis protocol then 2) a treatment protocol for the BALD syndrome.

The diagnostic protocol consists of 1) patient history; 2) clinical signs and symptoms and 3) corroborative tests and procedures.

Patient History:

The key is obtaining a history of exposure or possible exposure to environmental or industrial toxins or poisonous substances. Often there is the history of itching or burning of the scalp, shoulders or neck with possible neck and shoulder weakness which localizes dysfunction to the area of cervical segments C1 through C4 inclusive. There is often a history of numbness, weakness, or discomfort from peripheral neuropathy.

Signs of the CNS aspects of the BALD disorder are photo phobia and headache. Often there is a history of cognitive deterioration, memory problems, and insomnia. The patient will often experience occasional dizziness on standing, difficulty with walking straight, dizziness, difficulty with swallowing, neck weakness, an odd taste in the tongue usually tinny or metallic. There is usually decreased smell insensitivity for normal smells, but over reaction to very strong smells, and sometimes even olfactory hallucinations. Often there are accompanying gastrointestinal symptoms.

Clinical Signs and Symptoms:

Invariably the patient will experience abnormal increase in heart rate on standing, cranial nerve dysfunction, and in advanced cases mild extra pyramidal symptoms. The reflexes in the upper limbs are usually normal which distinguishes the disorder from other CNS disorders such as stroke and peripheral neuropathy. The reflexes and speed present at the knee are brisk, and other limb reflexes such as crossed adduction will be abnormal demonstrating the continued spreading of excess electrical excitability in the nervous system. Further, superficial pathological reflexes such as the glabellar, grasp reflex and finger flexion will appear. The normal infantile reflexes such as the tonic neck, placing reflex and crossed adductor reflex will re-appear. Multiple fasciculations appearing in muscles almost always signifies lower motor neuron dysfunction.

Step one in the clinical examination is to examine the sitting to standing heart rate. Usually this is not more than an increase in 10 to 15 beats per minute in the first few seconds after standing.

The stethoscope and a stop watch can be used or a pulse oximiter can be employed in this test. One check of the heart rate is made on sitting for 5 minutes, then after standing immediately a check is made at 5 seconds, a check at 15 seconds then a third check at 60 seconds. An abnormal increase on the sitting to standing heart rate indicates dysfunction in the nucleus of Cranial nerve X of the brainstem.

The next step in the clinical examination is to examine one or more of the cranial nerves. Dysfunction in a plurality of the cranial nerves is a strong indication that there is damage to the brainstem.

Examination of Cranial Nerve I the Olfactory Nerve:

With eyes dosed the patient is asked to identify mildly aromatic substances such as vanilla, cologne or cloves. If there is a disorder of Cranial Nerve I this indicates damage to the anterior part of the brainstem.

Examination of Cranial Nerve II the Optic Nerve:

The peripheral vision test is used. The patient is instructed to look straight ahead. Then an object is brought into the peripheral vision of the patient and the patient asked to state when the peripheral object is first seen. Loss of peripheral acuity results from damage to the optic tracts for this retinal area, as they course directly over the inflamed parts of the brain, particularly the cingulate gyrus. In the BALD disorder the upper outer quadrants of the peripheral vision are morer affected than the lower outer quadrants. Paleness of the optic disk or edema of the optic disk is looked for. A patient with inflammation to the brainstem and limbic system, will often experience low tolerance or intolerance to light shown in the eyes. Many cases of photophobia are subtle. The patient must be asked very carefully about increased use of dark glasses and hats, or avoidance of the outside. This involves the clinical experience of the practitioner.

Examination of Cranial Nerves III, IV, and VI, the Oculomotor Trochlear, and Abducens Nerves:

These three nerves are examined together, since they all act to control the extra ocular muscles. The patient is asked to blink as fast as possible. Fatigue of the levator muscles of the eyelids shows weakness in Cranial nerve III. A penlight is brought from a distance of several feet in front of the patient towards the eyes of the patient to test for visual convergence. The test is positive when there is diplopia up close. There can be observed by hyper convergence with double vision up close along with diplopia.

With lengthening of the penlight image distance horizontally, “sparkles” increase in the light of the penlight, or there is sudden darkening of the light or change in light color indicating cranial nerve dysfunction. These signs demonstrate loss of convergence at a distance. The light also is gradually moved backwards from the face, to twenty feet. At a distance a color change in the light can result. The most common color change is because of diffraction effects. At times the light will split into 2 lights at a distance. At other times, sparkles will appear or the patient will note the light elongating horizontally. As with other cranial nerves we will describe, there is reduced dynamic range of the reflex control of the cranial nerve responses. This is seen in Cranial Nerves 1, Ill, IV, V, VI, VII, and XII which all carry the special senses.

Examination of Cranial Nerve V, the Trigeminal Nerve:

This nerve carries sensory and motor neurons to specific areas of the face. The patient's facial sensation to pin prick, temperature, and vibration are tested. These are compared with the same sensations on the sternum. To test for damage to the third division of this nerve the three areas of the face and neck enervated by this cranial nerve are tested and compared for sensation. Sensory loss indicates damage to this nerve. Also small differences in sensation and delayed onset of sensory responses indicate damage to this nerve.

Examination of Cranial Nerve VII, the Facial Nerve.

This nerve is tested for damage by asking the patient to perform various facial movements and checking for loss of sensation inside of the external ear. The pupil of the eye may be wide. There may be fewer wrinkles on one side of the face, and there may be asymmetry to voluntary smiling or forehead wrinkling. Weakness of the nerve can be detected also by asking the patient to detect sweet, salty, and sour substances that are applied to the tongue. Loss of taste sensation definitely would indicate damage to this nerve. Examination of cranial nerve VIII the vestibulocochlear nerve.

This nerve transmits sound which allows a person to hear. Using a tuning fork the patient is asked to detect sound of lower frequencies from the tuning fork using air conduction and bone sound conduction. Loss of sound detection indicates damage to this nerve.

Examination of Cranial Nerves IX and X.

If cranial nerve IX is damaged the pharynx tonsillar pillars will show diminished sensitivity, especially on the side opposite of the greatest brainstem irritation. Cranial nerve X damage will cause a movement of the soft palate to the side, instead of the normal movement forward, when the tonsillar pillars are stimulated. Normally the soft palate should be symmetrical and should not deviate to either side. When speaking “me, la, ka from either side of the mouth, there may be a subtle deterioration of pronation, particularly on the side of the greatest brainstem irritation. Failure to properly enunciate these phonemes indicates damage to cranial nerve X. Further, these cranial nerves enervate the carotid sinus and the carotid bodies and damage to this nerve can be detected with the carotid body reflex which results in heart rate and blood pressure changes when moving from standing from a sitting position. The orthostatic tachycardia reflex which is reflex tachycardia resulting from change in position from lying to standing and which lasts only a few seconds can be used to detect damage to cranial nerve X. This reflex tachycardia can be best detected using a pulse oximeter. This cranial nerve controls the gastrointestinal tract, and gastroesophigeal reflex so that constipation and diarrhea are very frequent in the BALD syndrome.

Examination of Cranial Nerve XI, the Accessory Nerve.

This nerve is a motor nerve enervating the stemocliedomastoid muscle. To test this muscle for strength the patient is asked to turn the head toward one shoulder and to resist attempts of the examiner to move the head in the opposite direction. Then the test is repeated on the other side. Weakness in this muscle indicates damage to this cranial nerve. This nerve which moves the back of the head to the side being tested is the only cranial nerve with ipsilateral cortical connections.

Examination of Cranial Nerve XII, the Hypoglossal Nerve.

This nerve can be tested by asking the patient to push the tongue against either cheek then testing the strength of the tongue by pressing from the outside of the cheek. Fasciculation of the tongue, involuntary movements with the tongue at rest can be seen in advanced disease,

Spinal Cord Damage

The next step in the clinical examination is to test for upper spinal nerve damage. The strength of the trapezius muscle can be tested by asking the patient to push the muscle against the hands of the examiner. This checks for damage to spinal segments C-3 and C-4. Any weakness in the neck muscles especially on flexion of the head indicates weakness of the nerve damage to the brain stem and high cervical cord. Due to viral infection there may be shingles, or loss of sensation on spinal dermatomes, or in some cases weakness below the high cervical spine. There may be decreased strength of respiration, with shallow breathing, and difficulty with ventilation.

Motor Examination.

The next step In the clinical examination is to check for increased motor tone by moving joints in the arms over their passive range of motion and checking for involuntary muscle resistance. Spasticity or loss of fine motor cc-ordination indicates upper motor neuron damage. Increased motor tone indicates upper motor neuron damage, possibly damage to the brainstem and other brain structures in close proximity to the brainstem and limbic system.

Peripheral Nerve Examination:

Peripheral nerve damage using the pin prick test and temperature test to check for loss of sensation is useful. A cool tuning fork is handy for checking the limbs, as well as the face for temperature. There is often loss of two point discrimination especially on the side opposite the most affected part of the brainstem. In the advanced stages of the BALD disorder decreased sensation, altered sensation, and delay in experiencing sensation is observed.

In the BALD syndrome there is loss of stereognosis, so the patient cannot touch the area of skin the examiner has touched. There is, further, a loss of sensation or dyesthesea, abnormal, uncomfortable sensation on one or both sides of the limbs. There is loss of the ordinary ability to touch one's fingers together behind one's back with the eyes closed in the BALD syndrome. The normal person should be able to do this three times in a row without missing.

The next step is to check for abnormal reflexes, such as a mild g]abllar reflex. Other superficial reflexes are usually normal.

There is usually no Babinski sign. With localized damage to one side of the brainstem and limbic system there may be a partial Babinsky on the opposite side that is damaged.

Reappearance of Infantile Reflexes:

With brainstem damage infantile reflexes appear. The reflexes checked are: crossed extensor reflex, contra lateral reflex arc, deep tendon reflexes, the tonic neck reflex is checked for reappearance, and the infantile grasp reflex is check for reappearance. The infantile placing reflex is checked for extension of the leg muscles upon rubbing of the shin.

The clinical examination must check for lung and bronchial apparatus dysfunction. Prolonged expiration may be heard. The spirometry test is used and a number of these patients with brainstem and limbic system damage show mild restrictive airway disease. Increased auscultation usually points to increased intestinal motility.

The next step in the diagnosis protocol is to run laboratory tests to corroborate brain stem and limbic system damage. With neuro toxicity and damage to neurons there will he abnormal levels of evidence of viral and fungal infection. Specific, tests for abnormal levels of virus presence such as !he Barr-Epstein virus CMV virus, HHV6 virus, and HHV2 virus can show abnormal vulnerability to viral infections and re-infection. The T4/T8 lymphocyte levels can be tested for abnormality in the immune system. On the ususal lymphocyte panel tests used to identify immune system dysfunction there will be evidence of both immune suppression as well as evidence of auto immunity in the BALD syndrome. The T(4)/T(8) cell ratio is either too high or too low. The test for NADH is abnormal indicating neurotoxjcity and damaged neurons. In a nutshell abnormal results of tests for abnormal antibodies to neuron components reveals damaged neurons.

It is possible to run a coagulation panel or serum profile to be used to check for 1)fibrinogen antigen, 2) heparin assay, 3). thrombin/anti-thrombin complexes, 4) soluble fibrin monomer, and 5) platelet associated Ig G. Immune system activation of slow cold angulation and increased free fbrjn escaping in to the serum show immune system dysfunction and activation of coagulation, demonstrating a cause for tissue asphyxia or hypoxia. The root cause of this immune system dysfunction is brainstem dysfunction. Further the root cause of abnormal blood clotting is excitability of neurons and blood vessels from excessive intake of Calcium ions into the neuronal cells and blood vessel cells.

The next step in the diagnostic protocol is to run a MRI check with a long T-2 sequence, using Gadolinium, on the brainstem and limbic system. Necrotic tissue and gliosis can be observed in this test in severe cases of the BALD syndrome.

The next step in the diagnosis protocol is to run a brief neuropsychiatric examination checking for 1) loss of the sense of the familiar, 2) recent onset of obsessive behavior, 3) loss of predictability in behavior, and 4) decreased interpersonal involvement. Emotional disorders may be present such as hysterical responses to certain events.

The treatment protocol consists of ruling in or out of the BALD syndrome then treatment of the core pathology of the syndrome if present. This consists of treatment of the most acute aspects of the BALD syndrome followed by more conservative treatments of the more chronic symptoms or pathology. If diagnosis progresses to brainstem or other neuron damage with significant inflammation the treatment consists of immediate inflammation reduction and stabilization of the immune system. This is followed by conservative treatments as indicated in the diagnosis of the disorder.

The treatment proceeds in steps.

Step one in the treatment of the disorder would start with drugs that are known to block calcium intake channels of neurons. One such drug that has been used successfully clinically is Nimotop from Bayer Pharmaceutical Company. The chemical name for this drug is nimodipine which has the ability to inhibit movement of calcium ions across the cell membrane. Nimodipine has a greater effect on cerebral arteries than on other arteries, possibly because it is highly lipophilic. This is benefcial for a patient who is normotensive. The usual dosage can be 30 mg. three times daily more or less in the clinical judgment of the physician.

Another such drug is Plendil from Astra Merk Laboratories. The chemical name for this drug is feldipine which is known to also be a calcium intake channel blocking agent This is better for a patient who is hypertensive.

Step two in treatment of a severe disorder is to proceed with a drug that aids in inhibition of neural activity in the brainstem. The major inhibitory neurotransmitter in the central nervous system is gamma aminobutyric acid (GABA). Such a drug would be gabatril from Abbot Laboratories with a chemical name of tiagabine hydrochloride. It is believed that gabatril blocks GABA uptake into pre synaptic neurons, permitting more GABA to be available for receptor binding on the surfaces of post-synaptic cells. This exerts an anti seizure effect hy preventing the propagation of neural impulses that contribute to seizures by a GABA-ergic action. The GABA agonist drug such as gabatril or tigabine hydrochloride from Abbot Laboratories should be augmented by another drug Mysoline, an anti epileptic which increases the sensitivity of the GABA receptor neuron complex to GABA.

Step three in the treatment of a BALD syndrome involving viral infection to any degree is to administer anti-viral and anti-fungal drugs to the patient. Clinical success has been noted in the administration of Acyclovir which is a generic drug with a chemical name of acycloguanosine. This is a synthetic acyclic purine nucleoside analog. In vitro it has inhibitory activity against a broad spectrum of viruses such as herpes simplex virus types 1 and 2, varicella zoster virus, epstein-barr virus and cytomegalovirus. The drug inhibits viral DNA replication.

Another drug that has produced good clinical results in the BALD condition is difulcan from Roerig Pharmaceutical Company with a chemical name of fluconazole. This Is a synthetic broad spectrum bis-trazole antifungal agent. Still another clinically successful anti-viral drug from Glaxo Welcome is Valtrex with a chemical name of valacyclovir hydrochloride. Sproonox from Janssen is another drug that can be used successfully as an antifungal agent. This is a synthetic trazole antifungal agent with the chemical name of itraconazole.

Step four is to administer antibiotics to the patient to treat for streptococcal or other bacterial infection. Also mycoplasmal infection or parasitic infection should be treated. Antibiotics should be used with great caution keeping in mind that some antibiotics are themselves toxic and can damage already compromised tissues. It must always be understood that the BALD syndrome is a neurotoxic disease and is associated with tissue toxicity in general.

Step five is to treat immune disequilibrium and peripheral neuropathy by using Immune Globulin IV from human serum while the patient is still being administered GABA agonists and calcium intake channel blocking agents. This step should be initiated 3 to 6 months after ongoing treatment using steps 1 to 5. This is a generic drug which contains 5% immune globulins. The patient should be premedicated with benadryl from Parke Davis Pharmaceutical Company with a chemical name of diphenhydramine hydrochloride. Benadryl reduces or prevents most of the physiologic effects of histamine which includes inhibition of respiratory, vascular and gastro-intestinal smooth muscle constriction, decreased capillary permeability and decreased histamine activated exocrine secretions. Along with the use of immune globulin. IV, the patient should be given the generic drug methylprednisolone which is an adrenal glucococorticoid and which acts as a potent anti-inflammatory agent.

Step six in treatment involves administration of anti-coagulants to the patient. Heparin which is a generic drug can be used which inhibits body actions that lead to blood clotting. Other anti coagulant drugs such as coumarin may be needed as well.

Step seven is to administer a chelating agent to the patient to eliminate toxic heavy metals from the system. Clinical success has been achieved with the drug EDTA which is chemically knows as edate disodium. This drug Is readily displaced by heavy metals such as lead, to form stable complexes which can be excreted by the kidneys into the urine.

Step eight is to administer growth hormone to the patient to reduce cell death and to improve neovascularization of recovering tissue.

Step nine is to treat parallel affected organ systems. for example if there is a clinical finding of oxygen deficit, Oxygen and NADH should be administered to improve oxidation. For severe pain morphine sulphate and its analogs are best and are the first choice for analgesia and oxygen utilization. Morphine sulphate has good anti-inflammatory properties relative to neurons as well. Growth hormone can be used to protect against cellular destruction of tissues. Growth hormone releasing drugs if available can also be administered to the patient.

For any pulmonary problems, which are typically in the form of restrictive lung disease, common respiratory treatments with nebulizers are probably not helpful due to the irritating nature of typical medications used. Chromalyn by nebulizer is helpful.

Clinical success has ben achieved with Singulair from Merk Pharmaceutical with a chemical name of sodium montelukast. Also Accolate from Zeneca with a chemical name of zafirlukast is clinically successful. Singulair and Accolate are selective leukortine receptor antagonists which act to inhibit bronchoconstriction.

To improve oxygenation of the serum Oxygen by face mask or cannula can be admintered as well as NADH (ENADA) a generic substance to improve oxygenation. Heparin can also be administered to the patient along with Oxygen. If venous blood tests show partial pressure of Oxygen greater than 30 mm of mercury then Oxygen is not being properly absorbed through the arterial blood system.

The brainstem produces carbon monoxide in neuro transmission and this must be combined with serum Oxygen to produce Carbon Dioxide which is a much less harmful substance to neurons and other cells of the body.

This is often a very important part of the treatment protocol in the BALD syndrome.

Azulfadine should be started for patients with irritable bowel syndrome. This successful drug in clinical usage is from Pharmacia & Upjohn with a chemical name of sulfasalazine. This has an anti-inflammatory property to liver tissue and to tissue of the intestinal walls. This drug is indicated far ulcerative colitis. This should help considerably, especially if it is started at an interval of 3-5 days after the IV anti-microbial medication is given.

If there is persistent drooling, motor restlessness and cramping of the muscles, Amantadine Hydrochlyde can be administered.

Step nine in treatment involves treatment for pain, depression and psychosis. Numerous drugs work in this area depending on the symptotology involved. The first choice for analgesia is morphine sulphate a generic dnrg, with good anti-inflammatory properties relative to neurons.

Depression can be successfully treated with the generic drug trazedone hydrochloride. This is a monamine oxidase inhibitor which does not stimulate the central nervous system. In the central nervous system it selectively inhibits serotonin uptake by brain synaptosomes.

A clinically successful anti-anxiety drug is Klonapin with the chemical name of clonazepam. This drug also acts to prevent seizures.

Mag-ox can be administered for magnesium deficiencies and as an anti-acid:

All of the steps in the treatment of the BALD syndrome can be monitored by suitable clinical examination and laboratory tests.

The above description of the diagnostic protocol and the treatment protocol of the BALD syndrome is for purposes of illustration and not for purposes of limitation. The limitations of the present invention are set forth in the claims

What is claimed is:

1) A method of diagnosis of a disorder of the central nervous system of the human body that localizes the disorder to the brainstem of the body.

2) A method of diagnosis of a disorder of the central nervous system of the human body that localizes the disorder to the brainstem and limbic system of the body.

3) A method of diagnosis of a disorder of the human body that localizes the disorder to the brainstem and one or more other parts of the body.

4) A method of treatment of a disorder of the central nervous system of the human body that is localized to the brainstem of the body.

5) A method of treatment of a disorder of the brainstem of the body which also involves a disorder of at least one other part of the body.

6) A method of diagnosis indicated in claim 1 comprising the steps of:
clinical examination of the sitting to standing heart rate;
clinical examination of one or more of the twelve cranial nerves of the body;
clinical examination for upper spinal cord damage;
clinical examination for increased motor tone;
clinical examination for peripheral nerve damage;
clinical examination for abnormal reflexes;
using laboratory tests to check for infection. using laboratory tests to check for immune system disorder:
using laboratory tests to check for abnormal coagulation of the blood;
using laboratory tests to check for abnormal partial pressure of Oxygen in the venous blood;
using a mri test on the brainstem and limbic system; and
making a neuropsychiatric examination of the patient.

7) A method of diagnosis indicated in claim 1 comprising the steps of:
clinical examination of the sitting to standing heart rate;
clinical examination of one or more of the twelve cranial nerves of the body;
clinical examination for upper spinal cord damage;
clinical examination for increased motor tone;
clinical examination for peripheral nerve damage;
clinical examination for abnormal reflexes;
using laboratory tests to check for infection.
using laboratory tests to check for immune system disorder:
using laboratory tests to check for abnormal coagulation of the blood;
using laboratory tests to check for abnormal partial pressure of Oxygen in the venous blood;
using a mri test on the brainstem and limbic system; and
making a neuropsychiatric examination of the patient.

8) A method of diagnosis indicated in claim 3 comprising the steps of:
clinical examination of the sitting to standing heart rate;
clinical examination of one or more of the twelve cranial nerves of the body;
clinical examination for upper spinal cord damage;
clinical examination for increased motor tone;
clinical examination for peripheral nerve damage;
clinical examination for abnormal reflexes;
using laboratory tests to check for infection.
using laboratory tests to check for immune system disorder:
using laboratory tests to check for abnormal coagulation of the blood;
using laboratory tests to check for abnormal partial pressure of Oxygen in the venous blood;
using a mri test on the brainstem and limbic system; and
making a neuropsychiatric examination of the patient.

9) A method of diagnosis indicated in claim 3 comprising the steps of:
clinical examination of the sitting to standing heart rate;
clinical examination of one or more of the twelve cranial nerves of the body;
clinical examination for upper spinal cord damage;
clinical examination for increased motor tone;
clinical examination for peripheral nerve damage;
clinical examination for abnormal reflexes;
using laboratory tests to check for infection.
using laboratory tests to check for immune system disorder:
using laboratory tests to check for abnormal coagulation of the blood;
using laboratory tests to check for abnormal partial pressure of Oxygen in the venous blood;
using a mri test on the brainstem and limbic system; and
making a neuropsychiatric examination of the patient.

10) A method of diagnosis indicated in claim 3 comprising the steps of:
clinical examination of the sitting to standing heart rate;
clinical examination of one or more of the twelve cranial nerves of the body;
clinical examination for upper spinal cord damage;
clinical examination for increased motor tone;
clinical examination for peripheral nerve damage;
clinical examination for abnormal reflexes;
using laboratory tests to check for infection. using laboratory tests to check for immune system disorder:
using laboratory tests to check for abnormal coagulation of the blood;
using laboratory tests to check for abnormal partial pressure of Oxygen in the venous blood;
using a mri test on the brainstem and limbic system; and
making a neuropsychiatric examination of the patient.

11) A method of diagnosis indicated in claim 1 wherein one of the steps in the diagnostic procedure is
clinical examination of the sitting to standing heart rate;

12) A method of diagnosis indicated in claim 1 wherein one of the steps in the diagnostic procedure is
clinical examination of one or more of the twelve cranial nerves of the body;

13) A method of diagnosis indicated in claim 1 wherein one of the steps in the diagnostic procedure is
clinical examination for upper spinal cord damage;

14) A method of diagnosis indicated in claim 1 wherein one of the steps in the diagnostic procedure is
clinical examination for increased motor tone;

15) A method of diagnosis indicated in claim 1 wherein one of the steps in the diagnostic procedure is

16) A method of diagnosis indicated in claim 1 wherein one of the steps in the diagnostic procedure is
clinical examination for peripheral nerve damage;

17) A method of diagnosis indicated in claim 1 wherein one of the steps in the diagnostic procedure is
clinical examination for abnormal reflexes;

18) A method of diagnosis indicated in claim 1 wherein one of the steps in the diagnostic procedure is
using laboratory tests to check for infection.

19) A method of diagnosis indicated in claim 1 wherein one of the steps in the diagnostic procedure is
using laboratory tests to check for immune system disorder:

20) A method of diagnosis indicated in claim 1 wherein one of the steps in the diagnostic procedure is
using laboratory tests to check for abnormal coagulation of the blood;

21) A method of diagnosis indicated in claim 1 wherein one of the steps in the diagnostic procedure is
using laboratory tests to check for abnormal partial pressure of Oxygen in the venous blood;

22) A method of diagnosis indicated in claim 1 wherein one of the steps in the diagnostic procedure is
using a mri test on the brainstem and limbic system; and

23) A method of diagnosis indicated in claim 1 wherein one of the steps in the diagnostic procedure is
making a neuropsychiatric examination of the patient.

24) A method of diagnosis indicated in claim 2 wherein one of the steps in the diagnostic procedure is
clinical examination of the sitting to standing heart rate;

25) A method of diagnosis indicated in claim 2 wherein one of the steps in the diagnostic procedure is
clinical examination of one or more of the twelve cranial nerves of the body;

26) A method of diagnosis indicated in claim 2 wherein one of the steps in the diagnostic procedure is
clinical examination for upper spinal cord damage;

27) A method of diagnosis indicated in claim 2 wherein one of the steps in the diagnostic procedure is
clinical examination for increased motor tone;

28) A method of diagnosis indicated in claim 2 wherein one of the steps in the diagnostic procedure is

29) A method of diagnosis indicated in claim 2 wherein one of the steps in the diagnostic procedure is
clinical examination for peripheral nerve damage;

30) A method of diagnosis indicated in claim 2 wherein one of the steps in the diagnostic procedure is
clinical examination for abnormal reflexes;

31) A method of diagnosis indicated in claim 2 wherein one of the steps in the diagnostic procedure is
using laboratory tests to check for infection.

32) A method of diagnosis indicated in claim 2 wherein one of the steps in the diagnostic procedure is
using laboratory tests to check for immune system disorder:

33) A method of diagnosis indicated in claim 2 wherein one of the steps in the diagnostic procedure is
using laboratory tests to check for abnormal coagulation of the blood;

34) A method of diagnosis indicated in claim 2 wherein one of the steps in the diagnostic procedure is
using laboratory tests to check for abnormal partial pressure of Oxygen in the venous blood;

35) A method of diagnosis indicated in claim 2 wherein one of the steps in the diagnostic procedure is
using a mri test on the brainstem and limbic system; and

36) A method of diagnosis indicated in claim 2 wherein one of the steps in the diagnostic procedure is
making a neuropsychiatric examination of the patient.

37) A method of diagnosis indicated in claim 3 wherein one of the steps in the diagnostic procedure is
clinical examination of the sitting to standing heart rate;

38) A method of diagnosis indicated in claim 3 wherein one of the steps in the diagnostic procedure is
clinical examination of one or more of the twelve cranial nerves of the body;

39) A method of diagnosis indicated in claim 3 wherein one of the steps in the diagnostic procedure is
clinical examination for upper spinal cord damage;

40) A method of diagnosis indicated in claim 3 wherein one of the steps in the diagnostic procedure is
clinical examination for increased motor tone;

41) A method of diagnosis indicated in claim 3 wherein one of the steps in the diagnostic procedure is

42) A method of diagnosis indicated in claim 3 wherein one of the steps in the diagnostic procedure is
clinical examination for peripheral nerve damage;

43) A method of diagnosis indicated in claim 3 wherein one of the steps in the diagnostic procedure is
clinical examination for abnormal reflexes;

44) A method of diagnosis indicated in claim 3 wherein one of the steps in the diagnostic procedure is
using laboratory tests to check for infection.

45) A method of diagnosis indicated in claim 3 wherein one of the steps in the diagnostic procedure is
using laboratory tests to check for immune system disorder:

46) A method of diagnosis indicated in claim 3 wherein one of the steps in the diagnostic procedure is
using laboratory tests to check for abnormal coagulation of the blood;

47) A method of diagnosis indicated in claim 3 wherein one of the steps in the diagnostic procedure is
using laboratory tests to check for abnormal partial pressure of Oxygen in the venous blood;

48) A method of diagnosis indicated in claim 3 wherein one of the steps in the diagnostic procedure is
using a mri test on the brainstem and limbic system; and

49) A method of diagnosis indicated in claim 3 wherein one of the steps in the diagnostic procedure is
making a neuropsychiatric examination of the patient.

50) A treatment of a disorder of the human body as indicated in claim 4 comprising the steps of:
administration of one or more drugs to the patient that act so as to block the calcium intake channels of body cells;
administration of one or more drugs to the patient that act so as to aid in inhibition of neural activity in the human brain;
administration to the patient of at least one anti-viral drug;
administration to the patient of at least one anti-fungal drug;
administration to the patient of at feast one antibiotic drug;
administration to the patient of at least one substance to the patient that acts so as to provide immune system equilibrium to the body;
administration to the patient of at least one drug that acts so as to inhibit body actions that lead to blood clotting;
administration to the patient of at least one drug that acts as a chelating agent so as to remove toxic substances from the human body;
administration to the patient of growth hormone or a growth hormone releasing substance;
administration to the patient of Oxygen that will reach the arterial blood of the patient;
administration of modalities and drugs that treat parts of the human body other than the brainstem; and
administration to the patient of at least one drug that acts so as to relieve at least one symptom of psychosis;
then monitoring the treatment process with suitable clinical examination and laboratory tests.

51) A treatment of a disorder of the human body as indicated in claim 5 comprising the steps of:
administration of one or more drugs to the patient that act so as to block the calcium intake channels of body cells;
administration of one or more drugs to the patient that act so as to aid in inhibition of neural activity in the human brain;
administration to the patient of at least one anti-viral drug;
administration to the patient of at least one anti-fungal drug;
administration to the patient of at feast one antibiotic drug;
administration to the patient of at least one substance to the patient that acts so as to provide immune system equilibrium to the body;
administration to the patient of at least one drug that acts so as to inhibit body actions that lead to blood clotting;
administration to the patient of at least one drug that acts as a chelating agent so as to remove toxic substances from the human body;
administration to the patient of growth hormone or a growth hormone releasing substance;
administration to the patient of Oxygen that will reach the arterial blood of the patient;
administration of modalities and drugs that treat parts of the human body other than the brainstem; and
administration to the patient of at least one drug that acts so as to relieve at least one symptom of psychosis;
then monitoring the treatment process with suitable clinical examination and laboratory tests.

52) A treatment of a disorder of the human body as indicated in claim 4 wherein one of the steps is:
administration of one or more drugs to the patient that act so as to block the calcium intake channels of body cells;

53) A treatment of a disorder of the human body as indicated in claim 4 wherein one of the steps is:
administration of one or more drugs to the patient that act so as to aid in inhibition of neural activity in the human brain;

54) A treatment of a disorder of the human body as indicated in claim 4 wherein one of the steps is:
administration to the patient of at least one anti-viral drug;

55) A treatment of a disorder of the human body as indicated in claim 4 wherein one of the steps is:
administration to the patient of at least one anti-fungal drug;

56) A treatment of a disorder of the human body as indicated in claim 4 wherein one of the steps is:
administration to the patient of at feast one antibiotic drug;

57) A treatment of a disorder of the human body as indicated in claim 4 wherein one of the steps is:
administration to the patient of at least one substance to the patient that acts so as to provide immune system equilibrium to the body;

58) A treatment of a disorder of the human body as indicated in claim 4 wherein one of the steps is:
administration to the patient of at least one drug that acts so as to inhibit body actions that lead to blood clotting;

59) A treatment of a disorder of the human body as indicated in claim 4 wherein one of the steps is:
administration to the patient of at least one drug that acts as a chelating agent so as to remove toxic substances from the human body;

60) A treatment of a disorder of the human body as indicated in claim 4 wherein one of the steps is:
administration to the patient of growth hormone or a growth hormone releasing substance;

61) A treatment of a disorder of the human body as indicated in claim 4 wherein one of the steps is:
administration to the patient of Oxygen that will reach the arterial blood of the patient;

62) A treatment of a disorder of the human body as indicated in claim 4 wherein one of the steps is:
administration of modalities and drugs that treat parts of the human body other than the brainstem; and

63) A treatment of a disorder of the human body as indicated in claim 4 wherein one of the steps is:
administration to the patient of at least one drug that acts so as to relieve at least one symptom of psychosis;

64) A treatment of a disorder of the human body as indicated in claim 4 wherein one of the steps is:
then monitoring the treatment process with suitable clinical examination and laboratory tests.

65) A treatment of a disorder of the human body as indicated in claim 5 wherein one of the steps is:
administration of one or more drugs to the patient that act so as to block the calcium intake channels of body cells;

66) A treatment of a disorder of the human body as indicated in claim 5 wherein one of the steps is:
administration of one or more drugs to the patient that act so as to aid in inhibition of neural activity in the human brain;

67) A treatment of a disorder of the human body as indicated in claim 5 wherein one of the steps is:
administration to the patient of at least one anti-viral drug;

68) A treatment of a disorder of the human body as indicated in claim 5 wherein one of the steps is:
administration to the patient of at least one anti-fungal drug;

69) A treatment of a disorder of the human body as indicated in claim 5 wherein one of the steps is:
administration to the patient of at feast one antibiotic drug;

70) A treatment of a disorder of the human body as indicated in claim 5 wherein one of the steps is:
administration to the patient of at least one substance to the patient that acts so as to provide immune system equilibrium to the body;

71) A treatment of a disorder of the human body as indicated in claim 5 wherein one of the steps is:
administration to the patient of at least one drug that acts so as to inhibit body actions that lead to blood clotting;

72) A treatment of a disorder of the human body as indicated in claim 5 wherein one of the steps is:
administration to the patient of at least one drug that acts as a chelating agent so as to remove toxic substances from the human body;

73) A treatment of a disorder of the human body as indicated in claim 5 wherein one of the steps is:
administration to the patient of growth hormone or a growth hormone releasing substance;

74) A treatment of a disorder of the human body as indicated in claim 5 wherein one of the steps is:
administration to the patient of Oxygen that will reach the arterial blood of the patient;

75) A treatment of a disorder of the human body as indicated in claim 5 wherein one of the steps is:
administration of modalities and drugs that treat parts of the human body other than the brainstem; and

76) A treatment of a disorder of the human body as indicated in claim 5 wherein one of the steps is:
administration to the patient of at least one drug that acts so as to relieve at least one symptom of psychosis;

77) A treatment of a disorder of the human body as indicated in claim 5 wherein one of the steps is:
then monitoring the treatment process with suitable clinical examination and laboratory tests.
